(19) United States
(12) Patent Application Publication
(10) Pub. No.: US 2006/0216354 A1
(43) Pub. Date: Sep. 28, 2006

(54) Vector for oral administration

(75) Inventors: Yves Frere, Holtzheim (FR); Louis Danicher, Strasbourg (FR); Alain Belcourt, Strasbourg (FR)

Correspondence Address: YOUNG & THOMPSON, 745 SOUTH 23RD STREET, 2ND FLOOR, ARLINGTON, VA, 22202, US

(73) Assignee: CENTRE NATIONAL DE LA RECHERCHE SCIENTIFIQUE (C.N.R.S.), PARIS (FR)

(21) Appl. No.: 10/553,833

(22) PCT Filed: Apr. 20, 2004
(86) PCT No.: PCT/FR04/00974

§ 371 Date: Oct. 20, 2005

(30) Foreign Application Priority Data
Apr. 23, 2003 (FR) 03 04976

(51) Int. Cl. A61K 9/50; A61K 9/16

(52) U.S. Cl. 424/490; 977/906

(57) ABSTRACT

A vector for the oral administration of at least one pharmacologically active substance enabling the active substance to move from the intestinal lumen to the blood, optionally via intersticial liquid, without any substantial degradation of the substance, includes an essentially hydrophilic matrix having an outer surface which is modified by one or several chemical species providing the vector with an essentially lipophilic quality and containing one or several active substances. The vector is associated with a gastro-resistant vehicle enabling it to pass into the stomach without any denaturation and/or degradation of the active substance. The invention also relates to pharmaceutical compositions containing the vector. The vectors can be used to produce medicaments used in human or veterinary medicine.

The present invention relates to a vector intended for the oral administration of at least one pharmacologically active substance, to methods for preparing said vector, to uses thereof, and also to the pharmaceutical compositions that contain them.

Among the various routes of administration of ingredients that are active in the therapeutic field, oral administration and administrations by injection are by far the most commonly used. However, administrations by injection, which is an invasive method, are not always readily accepted by patients. In addition, many therapeutic treatments require several daily injections, which makes the treatment laborious and relatively inconvenient, in particular for patients who must permanently have the complete material for injection available to them and who, quite often, must perform their injections themselves.

Other routes of administration, such as nasal routes, pulmonary routes (of spray, aerosol, drop types, etc) or the like have often been found to be relatively ineffective in the therapeutic treatment of conditions other than nasal or pulmonary topical conditions themselves.

It would therefore be very advantageous to be able to as much as possible do away with nasal and pulmonary routes of administration, administration by injection, and the like, and to replace them with oral administrations, which are more physiological and more comfortable, for as many active ingredients as possible. Administration by injection has, however, well known advantages in this field, and in particular that of allowing the active ingredient to have a very rapid action since it is directly or virtually instantaneously available in the bloodstream.

Another considerable advantage of administration by injection is that many active ingredients cannot at this time be administered as such orally, said active ingredients being partially or totally degraded or denatured during ingestion (saliva, gastric juice, gastrointestinal enzymes, etc) before being able to reach the bloodstream. Such active ingredients are, for example, compounds that are peptide or protein in nature, such as vaccines, hormones (insulin, for example), and the like.

The advantages described above make it possible to explain why administrations by injection still remain at this time very widely used, to the detriment of oral administration. In fact, despite the very large number of studies carried out to date aimed at orally administering compounds sensitive to the conditions of the gastrointestinal tract (pH, mechanical stress, various enzymatic means), it has not been possible to satisfactorily combine the comfort of taking a medicament orally and the advantages of an injection (absence of denaturation or degradation, or small amount of denaturation or degradation of the active ingredient, rapid availability in the blood).

Many researchers have, however, attempted to solve these problems and the solutions revealed subsequent to these studies are diverse and varied. One of the main lines of research consists in protecting the active substances with a gastroresistant coating so as to decrease or even prevent denaturation, or even degradation, of the active substance when it passes into the stomach.

Thus, E. A. Hosny et al. (Pharmaceutica Acta Helvetiæ, 72, (1997), 203-207) have proposed to improve the availability of insulin administered orally by placing it in the presence of sodium cholate and protecting this mixture with coated capsules. The active substance is thus protected in the stomach and is released in the intestine. The results are, however, described only as “promising”: administration of capsules directly into the stomach of rats made hyperglycemic resulted in a blood glucose-lowering activity that was weaker than when an equivalent amount was administered by subcutaneous injection.

N. Shimono et al. (International Journal of Pharmaceutics, 245, (2002), 45-54) have envisaged using hydrophobic polymeric envelopes resistant to gastric attacks, such that the active substance is released specifically in the colon with the aim of treating conditions thereof. These envelopes are, consequently, resistant in the stomach and also in the small intestine, which appears to be a handicap in terms of the active substance reaching the bloodstream. This is because it appears necessary for the active substance to be released in the small intestine, and then to cross the intestinal wall in order to penetrate into the bloodstream, optionally via the interstitial fluid.

Another main line of research has been to determine pharmaceutical forms that make it possible to improve intestinal absorption of the active substance. In the publication by F. A. Dorkoosh et al. (International Journal of Pharmaceutics, 247, (2002), 47-55), systems based on superporous hydrogel polymers containing insulin, formulated as mini-lozenges, are described. Although oral administration is envisioned in those studies, no test was carried out using this route. In addition, the results presented, which are, however, incidentally not very reliable, do not make it possible to come to a conclusion as regards any convincing pharmacological effectiveness of the insulin administered by means of these systems.

Other publications present various pharmaceutical forms that can be orally administered, for example gelatin capsules, capsules (microcapsules or nanocapsules), matrix systems, or even less conventional systems such as “sponge” systems (R. Bodmeier et al., Pharmaceutical Research, 6(5), (1989), 413-417).

In oral administration, a third main line of research is aimed at increasing the amount of active substance absorbed through the intestinal barrier by adhesion of the compounds administered to the intestinal microvilli, i.e. mucoadhesion. For example, G. Ponchel et al. (European Journal of Pharmaceutics and Biopharmaceutics, 44 (1997), 25-31) have been interested in the mucoadhesion of carriers containing one or more active substances. However, the passing of these carriers through the intestinal epithelium is presented as being secondary. The improvement in bioavailability is only presented as resulting from the mucoadhesion and from the long period of adhesion time in the mucous membranes.

In U.S. Pat. No. 5,206,219, a pharmaceutical composition having an enteric coating suitable for oral administration, comprising a polyol pharmaceutical cosolvent combined with a lipid pharmaceutical solvent forming an emulsion on contact with the intestinal microvilli, is described. However, the problems of crossing the intestinal wall and of release of the active substance in the blood, optionally via the interstitial fluid, are not mentioned. Similarly, no mention is made of the bioassimilation of the components of this pharmaceutical composition.

Another solution has been described in the publication by G. P. Carrino et al. (Journal of Controlled Release, 65, (2000), 261-269), in which insulin coupled to zinc is encapsulated in nanospheres that have a poly(lactide-co-glycolide) coating, which nanospheres pass through the intestinal epithelium over a period ranging from 1 hour to 6 hours after oral administration. Since poly(lactide-co-glycolide) is a relatively hydrophilic polymer, the mucoadhesion to the intestinal villi is not satisfactory. Consequently, the authors added iron oxide in order to increase this mucoadhesion. However, even using such a system, the results obtained are far from being satisfactory, the pharmacological efficacy representing only 11.4% relative to the pharmacological activity of an equivalent amount of insulin administered intra-peritoneally.

Paradoxically, despite the number of studies carried out on the subject, no solution provided is truly satisfactory. There still remains today a need for oral administration systems.

Thus, a first objective of the invention consists in providing a system for oral administration of pharmacologically active substances that has characteristics of availability of said substances in the blood that are comparable to the characteristics of direct injection into the blood of said active substances.

Another objective consists in providing administration systems allowing the release in the blood of one or more pharmacologically active substances which are barely, or not at all, denatured or degraded during oral administration.

Another objective also consists in providing administration systems allowing one or more pharmacologically active substances to cross the intestinal wall without being substantially denatured or degraded.

One of the objectives also consists in providing administration systems allowing one or more pharmacologically active substances that have crossed the intestinal wall to be available in the blood, optionally after having passed into the interstitial fluid.

As another objective, the present invention consists in providing systems for the oral administration of one or more pharmacologically active substances, in which said active substance(s) are (is) barely, or not at all, denatured or degraded during passage into the gastrointestinal tract, and during passage across the intestinal wall, and which allow immediate, delayed or prolonged availability in the blood.

Other further objectives will become apparent in the description of the present invention that follows.

It has at present been discovered that the objectives described above can be achieved entirely or in part by means of administration vectors, such as those defined hereinafter.

Thus, and according to a first aspect, the present invention relates to a vector that is essentially lipophilic in nature and that allows the oral administration of at least one pharmacologically active substance, said substance being able to pass from the intestinal lumen to the blood, optionally via the interstitial fluid, without any substantial denaturation or degradation, the vector comprising a matrix that is essentially hydrophilic in nature and the outer surface of which is modified with one or more chemical species that give said vector an essentially lipophilic nature, and containing one or more active substances.

The oral administration of one or more active substances consists in fact in carrying the active substance(s) from the mouth to the blood, without said substances being substantially denatured or degraded. It is thus understood that the dose of active substance orally administered must be able to be substantially qualitatively and quantitatively found in the blood. The term “substantially quantitatively and qualitatively” is intended to mean that the amount of active substance in the blood relative to the amount of active substance orally administered must be greater than 50%, preferably greater than 65%, advantageously greater than 80%, optimally greater than 90%.

Thus, when administered orally, a pharmacologically active substance must overcome all the obstacles present in an organism before reaching the bloodstream, without undergoing any substantial denaturations or degradations. The main obstacles and difficulties, taken into account in the context of the present invention, are first of all the passage of the active substance in the stomach, the period spent in the intestinal lumen, the adhesion to the microvilli present in the intestine, and the passage from the intestine to the blood, optionally via the interstitial fluid.

The inventors considered firstly that the orally administered active substance(s) should reach the blood or the interstitial fluid without any substantial denaturation or degradation. As regards the interstitial fluid, it has a pH value of between approximately 6.5 and approximately 7.5, more particularly between approximately 7.2 and approximately 7.3, and has a not insignificant ionic strength.

Taking into consideration the fact that the characteristics of the blood and of the interstitial medium are different from the other organs (stomach, intestine, inter alia) that are passed from the buccal cavity, the inventors imagined a vector for the active substance, which vector may be biocompatible and bioassimilable or metabolizable.

Thus, this vector should be hydrophilic, so as to be compatible with the various body fluids (lymphatic fluid, interstitial fluid, blood, etc) . The vector should also release the active substance(s) rapidly, in a prolonged manner or in a delayed manner, at a pH of between approximately 6.5 and 7.5, ideally between approximately 7.2 and 7.3, so as to allow said substances to then be available in the blood, through the vector and/or after degradation thereof.

Without entering into complex mechanistic considerations, it is envisioned that the vector be degraded by the enzymes present in the environment (lysozyme, esterases, glycosidases, etc). The degradation of the vector will allow the immediate, prolonged or delayed release of the active substance(s) in the interstitial fluid so as to reach the bloodstream. Once in the blood, the active substance(s) will interact with the sites of interest, or will be transported to the sites or organs, in order to produce the desired pharmacological effect.

In addition, this vector must be optimized such that its hydrophilic nature is modified in order to make it compatible with the intestinal wall. This is because the intestinal wall is an essentially lipophilic environment, the pH of which is greater than approximately 7.8. It is, consequently, advisable to modify the vector described above such that it is essentially lipophilic in the region of and on the intestinal wall, that it exhibits relatively good mucoadhesion and, finally, that it withstands the enteric environment (basic medium of pH greater than approximately 7.8, presence of degradation enzymes, etc).

The inventors have discovered that it is possible to combine all the criteria mentioned above. To do this, a surface treatment is applied to a matrix that is hydrophilic in nature, comprising the active substance(s), so as to give it an essentially lipophilic nature. The combination of active substance(s), hydrophilic matrix and surface treatment giving the lipophilic nature defines the lipophilic vector according to the present invention.

The term “hydrophilic nature” or “essentially hydrophilic nature” is intended to mean a matrix that is solely hydrophilic in nature, or else lipophilic and hydrophilic in nature, the hydrophilic nature being, in this case, predominant with respect to the lipophilic nature in the medium of interest. Similarly, the term “lipophilic nature” or “essentially lipophilic nature” is intended to mean a vector that is solely lipophilic in nature, or else lipophilic and hydrophilic in nature, the lipophilic nature being, in this case, predominant with respect to the hydrophilic nature in the medium of interest. In the remainder of the present disclosure, it will be understood that, firstly, the terms “matrix that is (essentially) hydrophilic in nature” and “hydrophilic matrix” are equivalent and, secondly, that the terms “vector that is (essentially) lipophilic in nature” and “lipophilic vector” are equivalent.

The surface treatment defined above generally consists in modifying the surface of the hydrophilic matrix with one or more biocompatible chemical species capable of detaching therefrom when it passes from the intestinal lumen to the blood, optionally via the interstitial fluid.

The vector according to the invention is therefore a composite capable of satisfying all the criteria stated above, which comprises a hydrophilic matrix containing one or more active substances, the surface of which has been treated so as to give it a lipophilic nature.

The main constituent of the hydrophilic matrix of the vector is in general selected from polylactates, poly(lactate-co-glycolate)s (subsequently referred to as PLGAs), polymers or copolymers based on hyaluronic acid, on chitosan, on starch, on dextran and on the like, and also copolymers thereof and mixtures thereof. It may, for example, be envisioned that the main constituent of the matrix is a PLGA-hyaluronic acid, PLGA-chitosan, PLGA-starch or else PLGA-dextran mixture, or other mixtures.

Other constituents of the hydrophilic matrix can of course be envisioned, provided that they give said matrix an essentially hydrophilic and biocompatible and/or bioassimilable or metabolizable nature. These constituents should also be compatible with the active substance(s) contained in the vector, such that they are not substantially denatured or degraded before being available in the blood.

The matrix defined above is modified with chemical species capable of modulating its essentially hydrophilic nature in order to give it an essentially lipophilic behavior, compatible with its required properties of adhesion to the microvilli and of passage through the intestinal wall. Suitable chemical species are known to those skilled in the art, some of which are, for example, described by H. Takeuchi et al. (Adv. Drug Delivery Rev., 47, (2001), 39-54).

These chemical species are, for example, selected from paraffins, lecithins, amino acids, fatty acids in general and also derivatives thereof (esters and the like, for example stearates, glycerides), benzyls, inositol phosphates (Ips), glycerol phosphates, lipophilic polymers, and the like, and also mixtures thereof.

The outer surface of the hydrophilic matrix is subjected to a treatment with the chemical species defined above, which are thus attached to the hydrophilic matrix via “weak” bonds, such that said bonds can be detached from the matrix by contact with the microvilli present in the intestine and during the passage through the intestinal barrier. The detachment of the chemical species from the matrix thus allows the latter to return to its essentially hydrophilic nature.

The method for treating the surface of the hydrophilic matrix may be any type known in this field, that allows the adhesion, via one or more types of bonds defined above, of the chemical species to the outer surface of the hydrophilic matrix. A method of treatment may, for example, be of the type by soaking in a solution containing said chemical species, spraying of said substances, coating, film-coating, by cold-plasma treatment, etc. This surface treatment can also be carried out in such a way as to produce a monolayer- or multilayer-type coating.

The surface treatment of the matrix may be performed before or after the introduction of the active substance(s) into the matrix. In certain embodiments of the invention, for example when the matrix is in the form of a capsule membrane, the surface treatment can also be carried out during the preparation per se of the matrix.

After passage through the intestinal wall, and detachment of the chemical species, the matrix returns, via these means, to the hydrophilic nature required in the blood and/or the interstitial fluid, and which it had before treatment with the chemical species defined above. The weak bonds envisioned above can be of any type known to those skilled in the art and, for example, bonds of electrostatic and/or ionic nature and/or of hydrogen bond type, or the like.

It is also advisable for the vector (combination of active substance(s), matrix and chemical species) present in the intestinal lumen to be of a size and a shape that allows the physical passage of said vector across the intestinal membrane. In particular, the size of said vector will advantageously be between approximately 10 nm and approximately 10 μm, preferably between approximately 100 nm and approximately 500 nm, more preferably between approximately 200 nm and approximately 300 nm. A size of greater than 10 μm is less preferred since the vector would no longer be able to cross the intestinal wall. Similarly, a size of less than approximately 10 nm is equally less preferred, the amount of active substance transported by the vector possibly being too small.

The shape of the vector has no specific importance in itself, provided that it allows the latter to readily cross the intestinal wall. Thus, the vector may be in any known shape, for example sphere, needle, ovoid, etc, the largest dimension of which, a limiting factor for the passage through the intestinal wall, is advantageously between approximately 10 nm and approximately 10 μm, preferably between approximately 100 nm and approximately 500 nm, more preferably between approximately 200 nm and approximately 300 nm.

According to a preferred embodiment of the present invention, the vector is in the form of spheres having a diameter advantageously of between approximately 10 nm and approximately 10 μm, preferably between approximately 100 nm and approximately 500 nm, for example between approximately 200 nm and approximately 300 nm.

When the vector is in the form of spheres, said vector can be prepared according to conventional techniques for the encapsulation of active substances, such as, for example, by simple or complex coacervation, interfacial polycondensation, spray-drying, spray-coating, etc.

The vector according to the present invention comprises a matrix containing one or more pharmaco-logically active substances. In this respect, the matrix can be designed in the form of a gel containing an active substance, or several active substances in the form of a mixture. According to another aspect, the matrix is in the form of a capsule containing one or more active substances in the form of a mixture. Other forms can also be envisioned, for example “sponge”-type forms, or other solid forms that are more or less compact and are able to release, by diffusion and/or after degradation, the active substance(s) that they contain.

According to a preferred embodiment of the present invention, the vector is a capsule that is essentially lipophilic in nature, the membrane of which constitutes the hydrophilic matrix. The capsule contains one or more active substances or else a mixture of active substances, the membrane of the capsule having been modified with one or more chemical substances, thus giving it an essentially lipophilic nature.

It should be specified that, besides the active substance(s), the vector may also contain any appropriate excipient, filler, dye and the like, that are known to those skilled in the art and are not pharmacologically toxic.

The vector as defined above, comprising a hydrophilic matrix and containing one or more active substances, which matrix is modified with chemical species that give it a lipophilic nature, then has the characteristics that make it compatible with the intestinal medium. In particular, the lipophilic nature of the vector will ensure the property of mucoadhesion necessary for anchoring the vector onto the microvilli.

In order to be pharmacologically effective, this complex vector intended to be administered orally should also exhibit a high resistance to the stomach medium through which it will pass before reaching the intestine. The stomach is in fact an organ in which the pH is very acidic (in the region of 2, or even less). In addition, the enzymes present (in particular pepsin) in the stomach can denature, damage, or even completely destroy said vector and, by the same token, the active substance(s) that it contains.

Consequently, it is desirable to provide the vector defined above with gastric protection. The term “gastric protection of the vector” is intended to mean any carrier capable of protecting said vector against the physical stresses inherent in the stomach, these stresses being mainly the acidic pH and the stomach enzymes (pepsin). Of course, the constituents of the carrier, and also the denaturation or degradation products thereof, must be nontoxic for the organism.

Such carriers are already very widely known in the field (encapsulated medicaments for example, as described in “Encyclopedia of Pharmaceutical Technology”, Marcel Dekker, (1992), J. Swarbrick and J. C. Boylan Editors, Enteric Coatings, pp. 189-200). Any gastroresistant carrier known to those skilled in the art can, consequently, be used. Preferably, it may be solid in nature, in the form of a gel, or may be in the form of a coating or of a capsule, and may contain one or more carriers as defined above, themselves in various forms, capsules, gels or the like.

According to a preferred aspect of the present invention, the gastroresistant carrier is in the form of a capsule containing one or more vectors as defined above. Said carriers in the form of a capsule can advantageously be obtained by methods of the type coacervation, interfacial polycondensation in disperse medium, or the like. Of course, any other known method of encapsulation can be used and/or adapted with a view to preparing the carriers of the invention.

Among the constituents capable of withstanding the physiological stresses inherent in the stomach, mention may in particular be made of alginates, such as calcium alginate, carboxymethylcellulose, and the like, and also mixtures thereof. The gastroprotective carrier will have to withstand an acidic pH, in particular less than 2, and more particularly less than 1.2, and also attacks from the gastric enzymes.

Of course, the characteristics of the constituents of the carrier must include that of being able to be modified or degraded specifically in the intestinal lumen, i.e. at a pH of greater than approximately 7.8, and in the presence of the enteric enzymes, in order to release the vector in the intestinal lumen.

In addition, the gastroresistant carrier can optionally contain a lipophilic medium, in which the vector(s) defined above is (are) present. This lipophilic medium may be in solid or liquid form or else in the form of a gel. The lipophilic medium may consist of any lipophilic compound that is known in itself and pharmacologically nontoxic. The lipophilic compound envisioned may, for example, be selected from organic or mineral, plant or animal oils, for example olive oil, cod liver oil, silicon oils, and the like, and also mixtures thereof.

The vector comprising the hydrophilic matrix modified with chemical species that give it the lipophilic nature makes it possible to prevent any leaking of active substance, generally hydrophilic, into the intestinal stream, which is also hydrophilic. In addition to their mucoadhesive functions, the lipophilic modifications thus play a key role as a hydrophobic barrier to the active material.

Thus, the vector according to the present invention, provided with a gastric protection, may be useful for the oral administration of any pharmacologically active substance capable of being modified, affected, reorganized, metabolized, stored, denatured or degraded in the gastrointestinal tract, during a conventional direct oral administration. The gastro-resistant carriers comprising one or more vectors as have just been defined in the above description are also part of the present invention.

Another subject of the present invention concerns, consequently, the use of the vector according to the present invention for allowing pharmacologically effective oral administration of active substances that are peptide or protein in nature, i.e. that are characterized by one or more amino acid sequences. The active substances comprised in the vector of the present invention may thus be of diverse and varied nature.

By way of nonlimiting examples, mention may be made of hormones that are peptide in nature, and in particular insulin. The use of the vector according to the invention is not however limited to these active substances that are peptide or protein in nature, and any other pharmacologically active substance for which there is a risk of degradation or denaturation is also included in the field of the present invention.

As stated in the present description, the vector according to the invention will be used with a view to allowing the oral administration of one or more active substances, and in particular the transfer of said active substance(s) from the intestinal lumen to the bloodstream, optionally via the interstitial fluid.

The present invention also relates to the pharmaceutical compositions comprising one or more vectors, that may be identical or different, provided with at least one gastric protection, with any excipient, fillers, dyes, binders, other active substances, sweeteners, aromas, etc, that are nontoxic and well known to those skilled in the art.

Thus, and according to another subject of the present invention, the vector according to the present invention may be useful for the preparation of a medicament that can be administered orally in human or veterinary therapy and that has curative and/or preventive properties and/or properties that allow diagnosis, and in particular for the oral administration of active substances that are peptide or protein in nature, including vaccines.

The vector according to the invention finds, for example, an entirely advantageous use for preparing pharmaceutical products for oral administration in humans or animals. A particularly preferred use concerns the preparation of pharmaceutical products intended for various treatments, for example, and without implying limitation, those selected from the oral treatment of diabetes, and in particular of insulin-dependent Type 1 diabetes (vectorization of insulin), oral immunization (vectorization of vaccines), and hormone treatments (vectorization of hormones of any nature), to cite just a few nonlimiting examples.

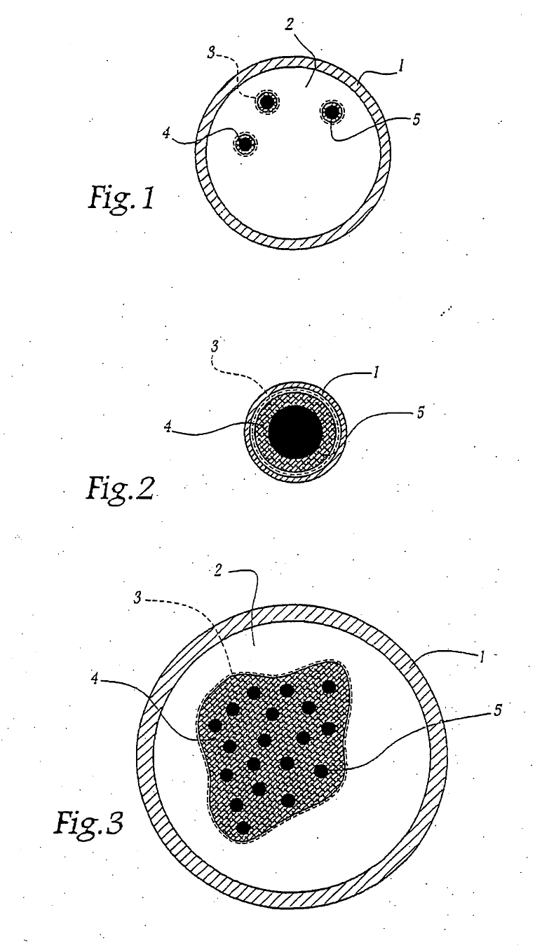

The attached FIGS. 1 to 3 present some nonlimiting examples of embodiment of the vectors according to the present invention.

FIG. 1 represents several vectors (3,4,5) dispersed in a lipophilic medium (2) and encapsulated in a gastric protection (1). Each vector (3,4,5) consists of an active substance (5) encapsulated in a hydrophilic matrix (4) modified with chemical species (3) that give said vector (3,4,5) the lipophilic nature.

FIG. 2 represents a vector (3,4,5) consisting of an active substance (5) encapsulated in a hydrophilic matrix (4) modified with chemical species (3) that give said vector (3,4,5) the lipophilic nature. The vector (3,4,5) is directly encapsulated in a gastric protection (1).

FIG. 3 represents a vector (3,4,5) consisting of a hydrophilic matrix (4) in the form of a gel in which an active substance (5) has been dispersed. This gel is modified with chemical species (3) that give said vector (3,4,5) the lipophilic nature. The vector (3,4,5) is placed in a lipophilic medium (2) which is itself encapsulated in a gastroresistant protection (1).

The following examples present possible embodiments for the present invention, without however limiting it in any way. It should also be understood that modifications can be introduced into these examples of embodiment, the vectors and carriers obtained remaining comprised within the field of the present invention.

### EXAMPLE 1

### Example of Synthesis of a Matrix in the Form of a Hydrophilic Capsule, Containing Insulin

Capsules are synthesized according to the multiple emulsion method with solvent extraction or evaporation.

The water-in-oil-in-water multiple emulsion is prepared in two steps:
in a first step, a water-in-oil simple emulsion is obtained by rapid dispersion (agitation: Ultra-Turrax® homogenizer; 15000 rpm; 3 times 10 s; 0° C.) of 50 μl of an aqueous solution of active ingredient (insulin; NovoRapid; 100 U/ml) in 5 ml of an organic solution (dichloromethane) containing 100 mg of biocompatible polymer (poly(L-lactate); Fluka; Mw=152000 or poly(D,L-lactate-co-glycolate); Aldrich; Mw=50000-75000; lactate/glycolate fraction=85/15 or poly(D,L-lactate-co-glycolate); Aldrich; Mw=50000-75000; lactate/glycolate fraction=50/50),
in a second step, this first emulsion is dispersed (agitation: Ultra-Turrax® homogenizer; 10000 rpm; 3 times 15 s; 0° C.) in 50 ml of an aqueous solution (1% w/v) of polyvinyl alcohol (Mowiol 4-88; Hoechst; Mw=26000).

In the case of the evaporation method, the multiple emulsion is diluted in 150 ml of an aqueous solution (0.3% w/v) of polyvinyl alcohol (Mowiol 4-88; Hoechst; Mw=26000), and this solution is stirred under reduced pressure (rotary evaporator; 500 mm/Hg; 3 h; 25° C.) so as to allow evaporation of the organic solvent.

In the case of the extraction method, the multiple emulsion is diluted in 200 ml of an aqueous solution (2% v/v) of isopropanol, and this solution is stirred (agitation: magnetic; 250 rpm; 1 h; 25° C.) so as to allow extraction of the organic solvent.

After evaporation or extraction of the organic solvent, the capsules are recovered by filtration, washed with water, centrifuged, lyophilized, and then stored in the cold (4° C.).

### EXAMPLE 2

### Example of Synthesis of a Vector Consisting of a Matrix in the Form of a Sponge Comprising Insulin

“Sponge”-type matrices are synthesized according to the complex coacervation method.

Two aqueous solutions, one (5 ml; 1% w/v in water) a solution of sodium hyaluronate (from Streptococcus Equi; Fluka), the other (5 ml; 1% w/v in 0.1N acetic acid) a solution of chitosan (from crab shells; Fluka; Mw=150000), are dispersed (agitation: Ultra-Turrax® homogenizer; 15000 rpm; 0°0 C.) simultaneously but separately in 100 ml of an organic phase (mineral oil; Aldrich) containing a surfactant (Span®80; Aldrich; 1% w/v).

The addition is carried out by means of a syringe (inside diameter of the needle: 0.6 mm) and of a syringe pump (0.2 ml/min).

The dispersion is then stirred (magnetic stirring; 200 rpm; 12 h; 40° C.) so as to allow sponge formation.

The particles are separated from the organic phase by centrifugation, washed with cyclohexane (3 times 100 ml), filtered, and then lyophilized.

The sponges are immersed (12 h; 25° C.) in an aqueous solution of active ingredient (insulin; NovoRapid; 100 U/ml), filtered, rinsed rapidly with sterilized water, lyophilized, and then stored in the cold (4° C.).

The sponges (that are essentially hydrophilic in nature) are soaked in a solution of fatty acids so as to obtain vectors that are essentially lipophilic in nature.

### EXAMPLE 3

### Example of Synthesis of Carriers Containing the Vectors of Example 2

The carriers are synthesized according to the coacervation method.

The vectors (100 mg) obtained in example 2 and containing the active ingredient (insulin; NovoRapid; 100 U/ml) are hydrated, filtered, and then dispersed (stirring: magnetic; 200 rpm; 5 min) in an organic phase (olive oil; 1 ml) with calcium carbonate reduced to fine powder (100 mg).

This organic phase is added by means of a syringe (inside diameter of the needle: 1.2 mm) and of a syringe pump (0.2 ml/min), to 50 ml of an aqueous phase (0.25% w/v) of sodium alginate (Lancaster) and (1% v/v) of acetic acid, stirred continuously (stirring: mechanical (propeller); 300 rpm; 1 h; 25° C.).

The alginate solution is diluted by adding 200 ml of deionized water. The carriers are recovered by filtration and transferred into an aqueous calcium chloride solution (1.3% w/v). After incubation at ambient temperature for 15 min, the carriers are filtered, rinsed with deionized water, and then stored in water in the cold (4° C.).

1. A vector for the oral administration of at least one pharmacologically active substance, that allows said active substance to pass from the intestinal lumen to the blood, optionally via the interstitial fluid, without any substantial denaturation or degradation of said substance, the vector comprising a matrix that is essentially hydrophilic in nature, the outer surface of which is modified with one or more chemical species that give said vector an essentially lipophilic nature, and containing one or more active substances.

2. The vector as claimed in claim 1, characterized in that it is biocompatible and bioassimilable or metabolizable at a pH of between approximately 6.5 and 7.5, ideally between approximately 7.2 and 7.3.

3. The vector as claimed in claim 1, characterized in that the chemical species are detached from the matrix when the vector passes from the intestinal lumen to the blood, optionally via the interstitial fluid.

4. The vector as claimed in claim 1, characterized in that the main constituent of the hydrophilic matrix is selected from polylactates, poly(lactate-co-glycolate)s, polymers or copolymers based on hyaluronic acid, on chitosan, on starch, on dextran and the like, and also copolymers thereof and mixtures thereof.

5. The vector as claimed in claim 1, characterized in that the chemical species are selected from paraffins, lecithins, amino acids, fatty acids in general and also derivatives thereof (esters and the like, for example stearates, glycerides), benzyls, inositol phosphates (IPs), glycerol phosphates, lipophilic polymers, and the like, and also mixtures thereof.

6. The vector as claimed in claim 1, characterized in that the chemical species are attached to the hydrophilic matrix via weak bonds.

7. The vector as claimed in claim 6, characterized in that the weak bonds are bonds of electrostatic and/or ionic nature and/or of hydrogen bond type.

8. The vector as claimed in claim 1, characterized in that its largest dimension is between approximately 10 nm and approximately 10 μm, preferably between approximately 100 nm and approximately 500 nm, more preferably between approximately 200 nm and approximately 300 nm.

9. The vector as claimed in claim 8, characterized in that it is in the form of spheres having a diameter of between approximately 10 nm and approximately 10 μm, preferably between approximately 100 nm and approximately 500 nm, for example between approximately 200 nm and approximately 300 nm.

10. The vector as claimed in claim 1, characterized in that it comprises a matrix in the form of a gel containing said active substance(s) or else a mixture of active substances.

11. The vector as claimed in claim 1, characterized in that it comprises a matrix in the form of a capsule containing said active substance(s) or else a mixture of active substances.

12. The vector as claimed in claim 1, characterized in that it has gastric protection.

13. The vector as claimed in claim 12, for which the gastric protection is solid in nature, in the form of a gel, or is in the form of a coating or of a capsule.

14. The vector as claimed in claim 13, for which the gastric protection is in the form of a capsule.

15. The vector as claimed in claim 12, characterized in that the gastric protection comprises constituents selected from alginates, such as calcium alginate, carboxymethylcellulose and the like, and also mixtures thereof.

16. The vector as claimed in claim 1, characterized in that it has gastric protection containing said vector in a lipophilic compound.

17. The vector as claimed in claim 16, characterized in that the lipophilic compound is selected from organic or mineral, plant or animal oils, and mixtures thereof.

18. The vector as claimed in claim 1, consisting of a plurality of hydrophilic capsules modified with chemical species that give them a lipophilic nature, said capsules being dispersed in a lipophilic medium that is itself contained in a capsule that acts as gastric protection.

19. The vector as claimed in claim 1, characterized in that the active substance is selected from substances capable of being denatured or degraded upon direct oral administration.

20. The vector as claimed in claim 1, characterized in that the active substance is peptide or protein in nature.

21. The vector as claimed in claim 1, characterized in that the active substance is insulin.

22. A gastroresistant carrier comprising one or more vectors as claimed in claim 1.

23. A pharmaceutical composition comprising at least one vector as defined in claim 1 or a gastroresistant carrier comprising at least one said vector.

24. Method of preparing a medicament that is active when administered orally in human or veterinary therapy and that has curative and/or preventive properties and/or properties that allow diagnosis, which comprises using an effective amount of a vector as claimed in claim 1 with an appropriate excipient.

25. The Method as claimed in claim 24, for producing a pharmaceutical product intended for the treatment of Type 1 diabetes.

26. The Method as claimed in claim 24, for producing a pharmaceutical product intended for oral immunization.
